(19) United States
(12) Patent Application Publication
(10) Pub. No.: US 2025/0143567 A1
(43) Pub. Date: May 8, 2025

(54) METHOD AND SYSTEM FOR THE DETECTION OF CEREBRAL AMYLOID ANGIOPATHY

(71) Applicant: Steven Verdooner, Sacramento, CA (US)

(72) Inventor: Steven Verdooner, Sacramento, CA (US)

(73) Assignee: NeuroVision Imaging, Inc., Sacramento, CA (US)

(21) Appl. No.: 18/940,096

(22) Filed: Nov. 7, 2024

Related U.S. Application Data
(60) Provisional application No. 63/596,962, filed on Nov. 7, 2023.

(51) Int. Cl. A61B 3/10; A61B 3/00; A61B 3/14; A61B 5/00; G06T 7/00; G06T 7/10; G16H 50/20

(52) U.S. Cl. CPC A61B 3/102; A61B 3/0025; A61B 3/1025; A61B 3/14; G06T 7/0012; G06T 7/10; A61B 3/1241; G06T 2207/10064; G06T 2207/20024; G06T 2207/20036; G06T 2207/20076; G06T 2207/30041

(57) ABSTRACT

A method and system detects cerebral amyloid angiopathy (CAA) condition of a subject by capturing multiple images of the subject's retina, processing the images to detect any retinal hemorrhage, retinal vessel tortuosity, and retinal edema, to predict the likelihood of CAA.

### CROSS-REFERENCE TO RELATED APPLICATIONS

This application claims priority to U.S. Provisional Application No. 63/596,962 filed on Nov. 7, 2023, incorporated by reference herein.

### BACKGROUND OF THE INVENTION

Cerebral amyloid angiopathy (CAA) is a neurological condition characterized by the accumulation of amyloid proteins in the walls of the blood vessels in the brain. Specifically, it's the amyloid beta-peptide that is commonly deposited. This peptide is also known for its association with Alzheimer's disease, where it accumulates in the parenchyma of the brain to form plaques. In CAA, however, the buildup occurs within the walls of small to medium-sized cerebral arteries, arterioles, and, sometimes, capillaries and veins.

### Clinical Features and Risks:

Hemorrhagic Stroke: The primary clinical concern with CAA is an increased risk of hemorrhagic stroke due to the weakening of the blood vessel walls. This can lead to lobar intracerebral hemorrhage, which typically occurs in the outer (cortical) areas of the brain.

Cognitive Impairment: While CAA can be asymptomatic, it has been associated with cognitive decline and dementia in some patients.

Microbleeds: It is also often associated with cerebral microbleeds, which may be seen on MRI scans as areas of hemosiderin deposition due to previous small hemorrhages.

Leukoaraiosis: The condition may also lead to white matter changes in the brain, known as leukoaraiosis, which can be detected through imaging.

### Pathogenesis:

The pathogenesis of CAA involves the deposition of amyloid in the media and adventitia of cerebral vessels, leading to vessel wall fragmentation and loss of structural integrity. This process can cause the vessels to become brittle and prone to rupture or lead to compromised blood flow to certain brain regions.

### Diagnosis:

MRI: Magnetic resonance imaging, especially gradient-echo sequences or susceptibility-weighted imaging, can reveal hemosiderin deposits indicative of previous microhemorrhages.

Histopathological Examination: Definitive diagnosis often requires a biopsy or post-mortem examination, where the presence of amyloid can be confirmed in the vessel walls with special staining techniques.

Clinical Correlation: It's also important to correlate imaging findings with clinical presentation, as similar imaging features can be caused by other conditions.

### Treatment and Management:

There is no definitive cure for CAA. Treatment typically focuses on managing symptoms and reducing risk factors for stroke:

Blood Pressure Control: Keeping blood pressure in check is crucial, as hypertension can increase the risk of hemorrhage.

Avoiding Anticoagulants: If possible, avoiding anticoagulation therapy is recommended since it can increase the risk of bleeding in patients with CAA.

Supportive Care: For patients who have suffered a hemorrhage, supportive care and rehabilitation may be necessary.

### Research:

Research into CAA is ongoing, particularly into its relationship with Alzheimer's disease and other dementias, and into potential treatments that can target amyloid deposition.

As CAA is a significant cause of cerebral hemorrhage in the elderly, it remains an important area of study in neurology and geriatric medicine.

Retinal autofluorescence imaging is a non-invasive diagnostic technique used in ophthalmology to assess the health of the retina. This method utilizes the natural fluorescent properties of certain compounds within the eye to visualize and monitor retinal disorders.

### Principles of Autofluorescence Imaging (AF):

Autofluorescence: Certain components within the retina, particularly lipofuscin and amyloid, naturally emit fluorescence when stimulated by specific wavelengths of light. Lipofuscin is a byproduct of the visual cycle and accumulates within the ribulose 5-phosphate 3-epimerase (RPE) cells over time. Amyloid in the retina refers to the accumulation of abnormal protein deposits known as amyloid. These are the same type of protein aggregates found in other parts of the body in various diseases, most notably in Alzheimer's disease within the brain.

In the context of the retina, the presence of amyloid deposits can be an indication of underlying pathology. These deposits can disrupt the normal function of the retinal cells and may be associated with retinal degeneration or systemic diseases like Alzheimer's. While not as common as in other tissues, the detection of retinal amyloid deposits can potentially serve as a biomarker for neurodegenerative diseases and might one day aid in early diagnosis.

Excitation and Emission: During autofluorescence imaging, retinal areas are illuminated with blue or green light which excites the molecules. These molecules then emit light at a higher wavelength that can be captured by the imaging device.

### Clinical Uses:

Diagnosis: Autofluorescence can help in the diagnosis of various retinal conditions, including age-related macular degeneration (AMD), inherited retinal dystrophies, central serous chorioretinopathy, and more.

Monitoring Disease Progression: The presence, absence, or patterns of autofluorescence can indicate the health of the RPE and photoreceptor cells, thereby helping to monitor the progression of retinal diseases. Furthermore, these patterns can indicate and predict the presence of amyloid in the brain, including along blood vessels as is the case with CAA.

Guiding Treatment: Autofluorescence imaging can guide the application of certain treatments, such as identifying areas for laser photocoagulation or anti-vascular endothelial growth factor (VEGF) therapy.

### Procedure:

Pupil Dilation: The patient's pupils are usually dilated to allow a better view of the retina.

Imaging: The patient is seated in front of the autofluorescence imaging device, and a series of photographs is taken.

Interpretation: Variations in the autofluorescence pattern can indicate different types of retinal conditions. Increased autofluorescence may indicate an accumulation of lipofuscin or other fluorophores, whereas decreased autofluorescence may suggest a loss of RPE cells.

### Advantages of Retinal Autofluorescence Imaging:

Non-invasive: The technique does not require injections of contrast agents, making it very safe and easy to perform.

Quick: The imaging process is relatively quick, which is beneficial for patient comfort and clinical workflow.

Informative: It provides valuable information about the metabolic state of the RPE and the overlying photoreceptor cells.

### Limitations:

Interpretation: Interpretation of autofluorescence images requires experience and can be challenging because the fluorescence signals may be affected by a variety of factors, including the density of the RPE, the presence of media opacities, and the concentration of lipofuscin.

Resolution: While autofluorescence imaging is quite sensitive, it may not capture all the subtle changes or early signs of retinal diseases. Single image capture AF images do not correlate with retinal histology due to low signal in the in vivo images.

Currently there are no biomarkers for CAA other than the Boston and Boston 2 Criteria which rely on MRI, and only in advanced stages of disease. There are not fluid or imaging biomarkers for CAA.

### SUMMARY OF THE INVENTION

The present invention addresses problems of the prior art through novel image capture, image processing, and machine/deep learning techniques that allow for significant boost in image quality and the ability to quantify:
Perivascular amyloid accumulation in and around retinal blood vessels
Retinal vessel tortuosity.
Retinal hemorrhage
Retina edema

One or more combinations of this data is predictive of CAA.

### BRIEF DESCRIPTION OF THE DRAWINGS

FIG. 1 is a block diagram illustrating the system architecture and image processing workflow of the invention, showing key processing steps including image quality assessment, noise filtering, image combination, enhancement, region of measurement definition, and feature identification;

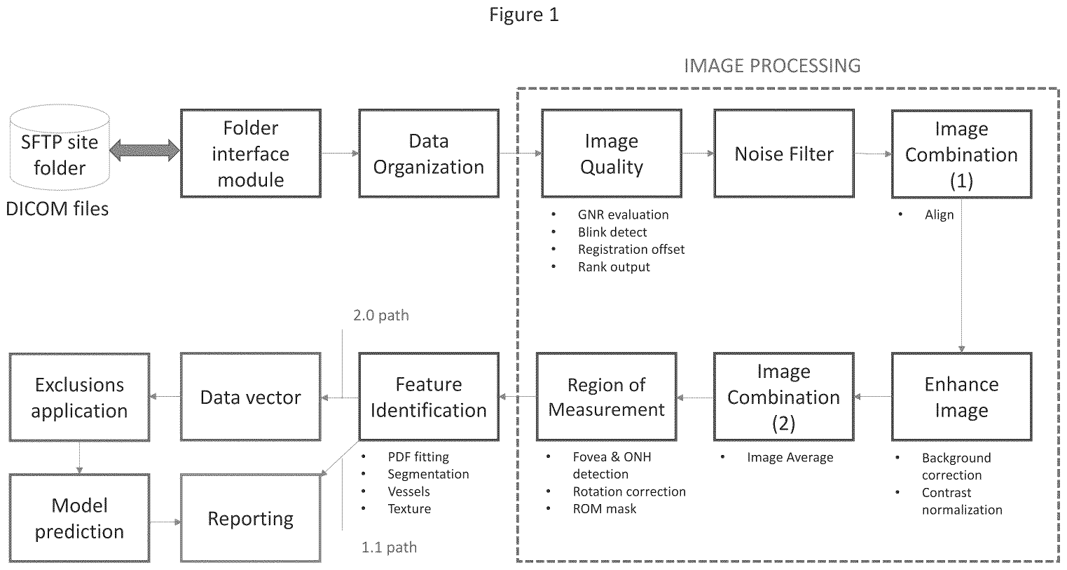

FIG. 2 illustrates the concept of brain amyloid beta assessment using retinal imaging;

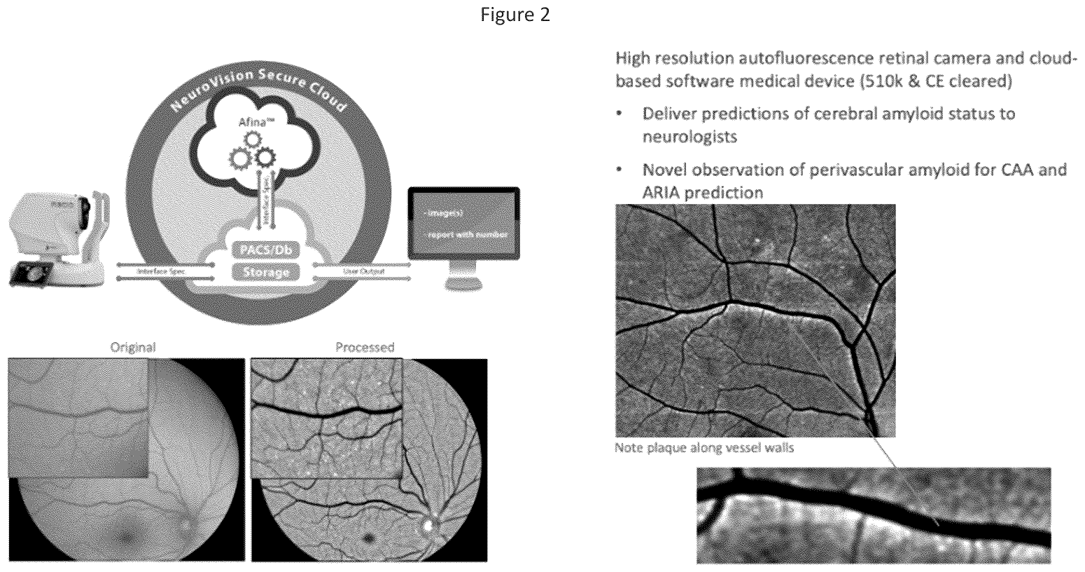

FIG. 3 shows data demonstrating intense deposition of retinal vascular Aβ40 in mild cognitive impairment (MCI) and Alzheimer's Disease (AD) patients correlated with CAA severity;

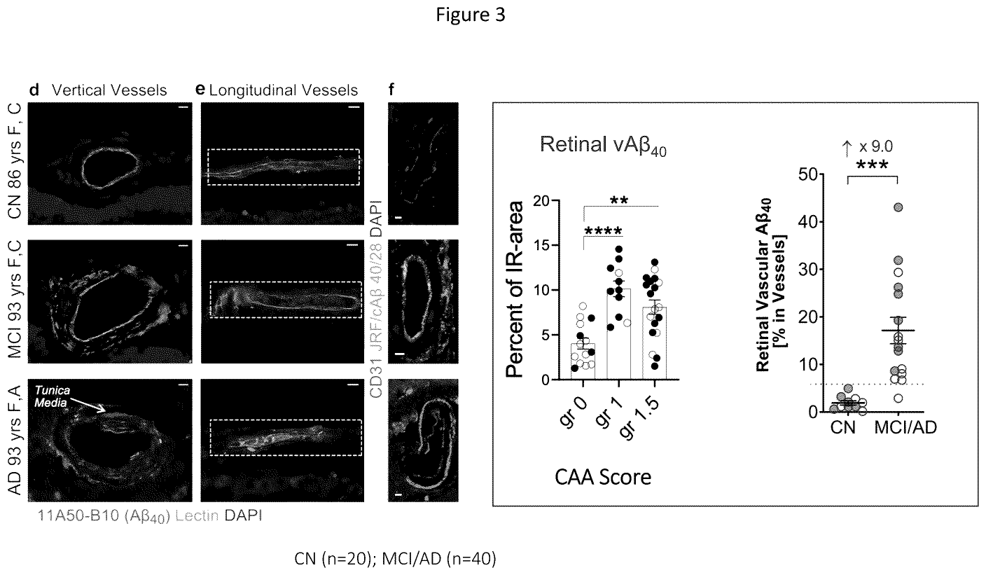

FIG. 4 presents the methodology and parameters of the Kuopio Study clinical dataset;

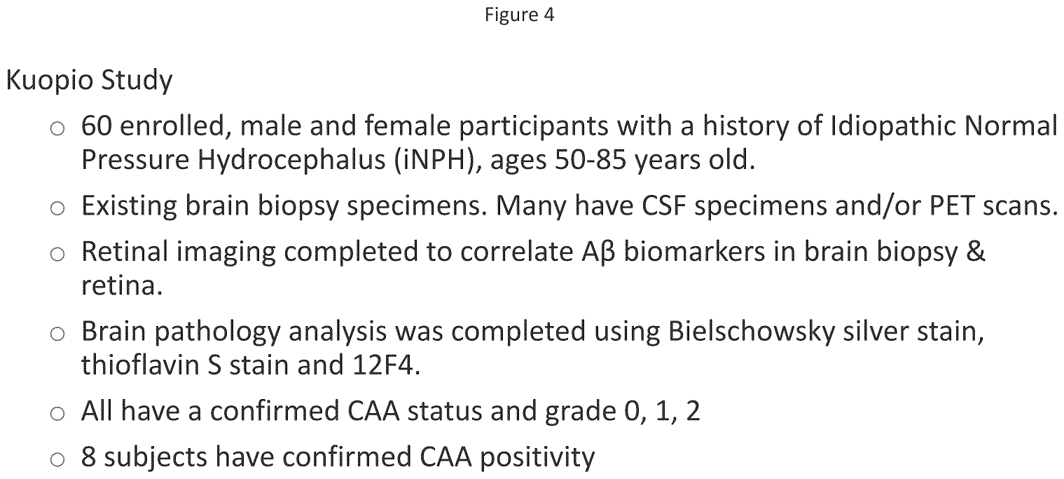

FIG. 5 presents detailed data analysis from the Kuopio Study focusing on CAA+ subjects;

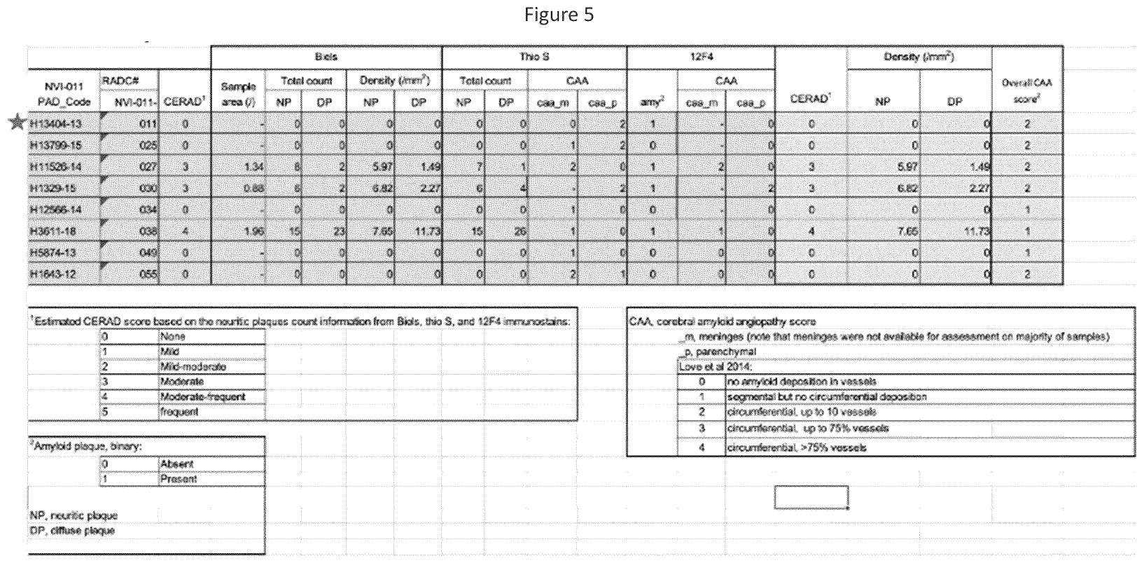

FIG. 6 shows representative histological images from a CAA+ subject;

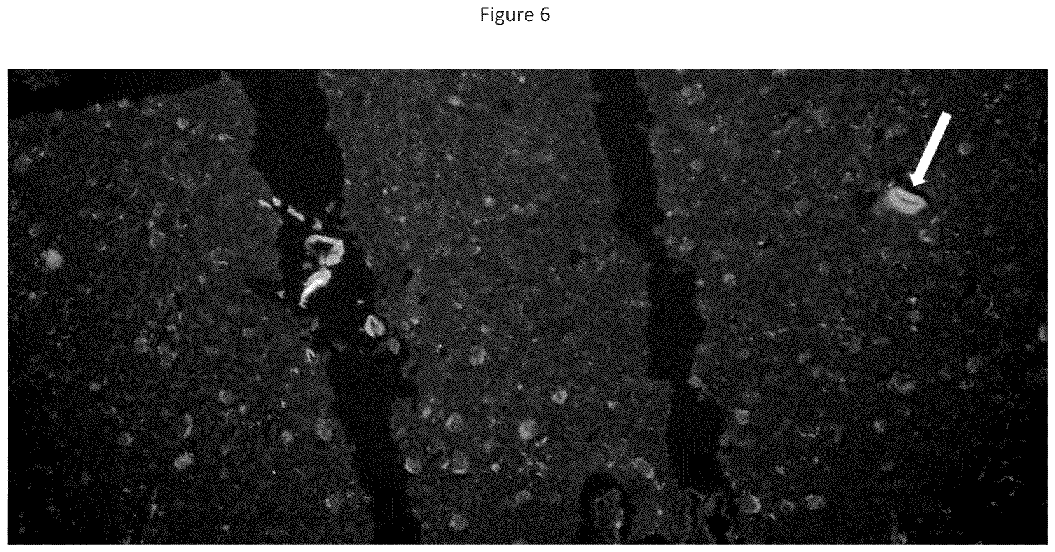

FIG. 7 demonstrates the automated vessel map generation process, comparing processed images (left) with deep learning-generated vessel maps (right);

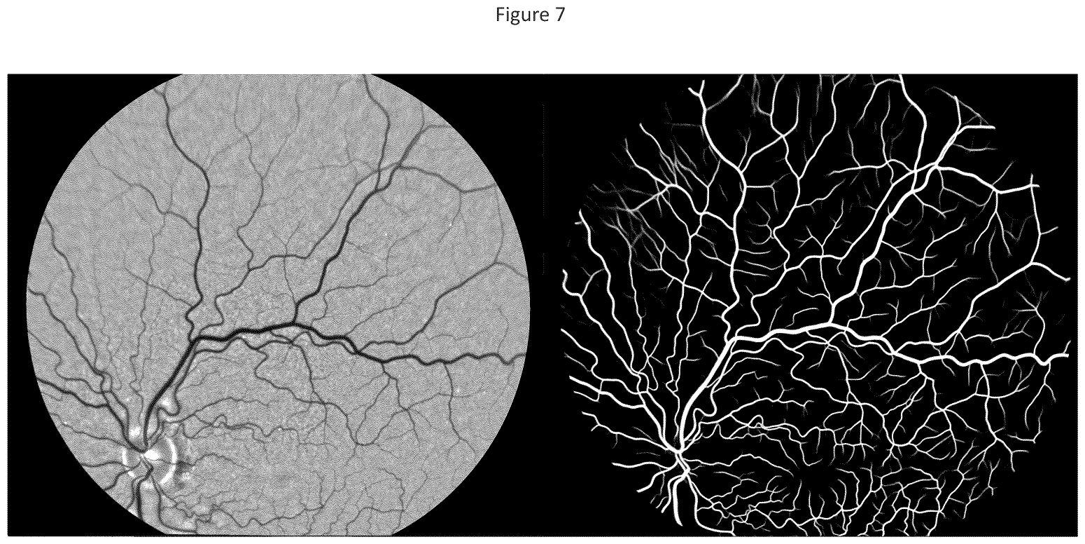

FIG. 8 presents imaging results from a histologically confirmed CAA+ subject, showing processed (left) and final segmented (right) images;

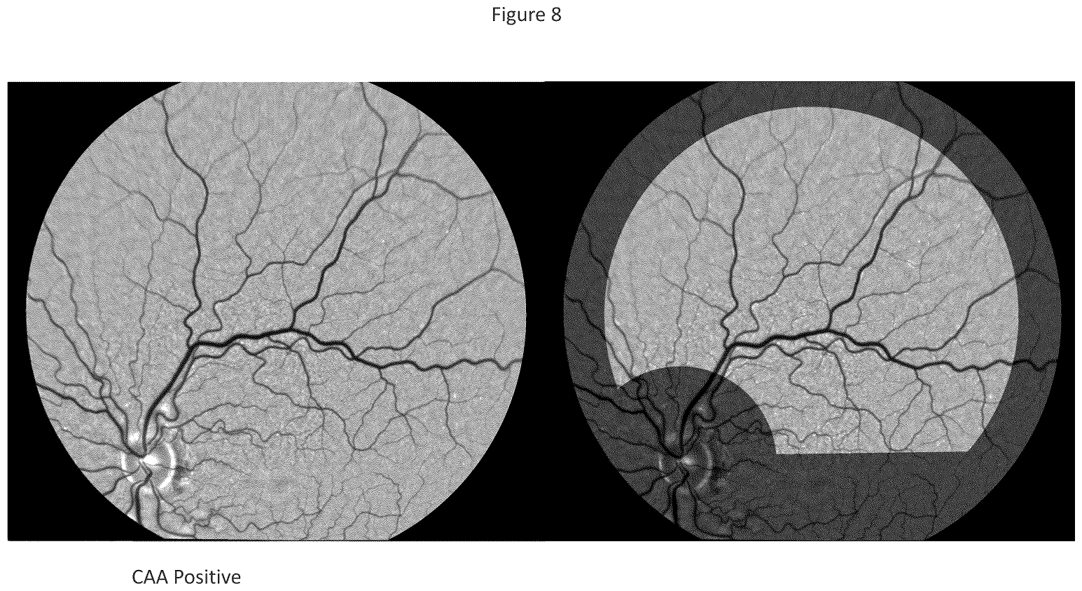

FIG. 9 presents imaging results from a histologically confirmed CAA− subject, showing processed (left) and final segmented (right) images;

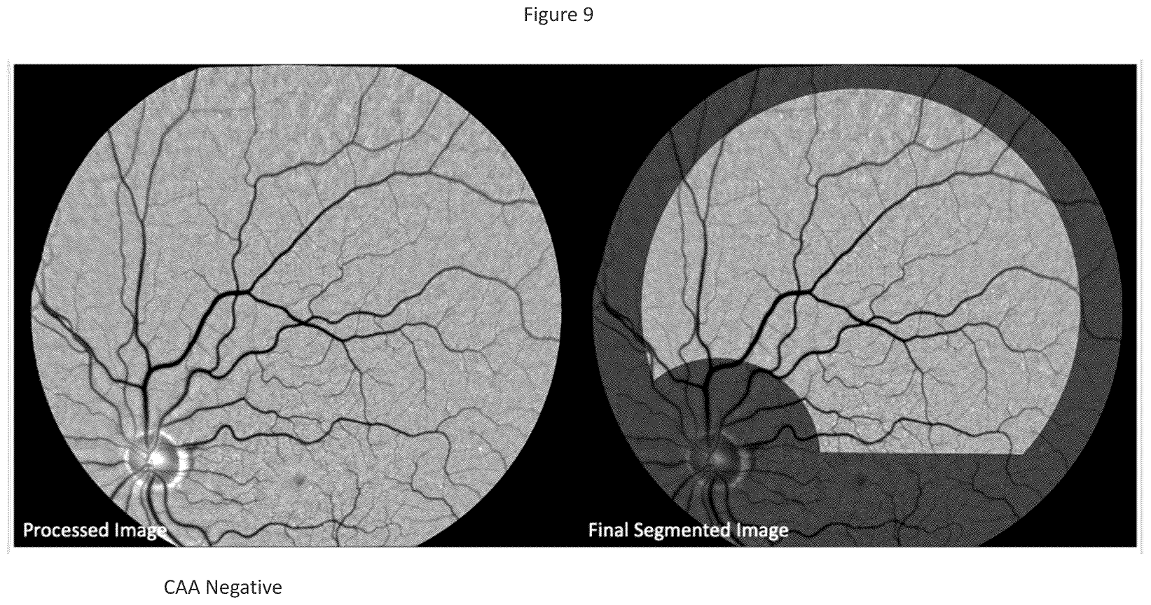

FIG. 10 illustrates perivascular amyloid accumulation in a CAA+ subject, with magnified inset showing preferential accumulation along arteries;

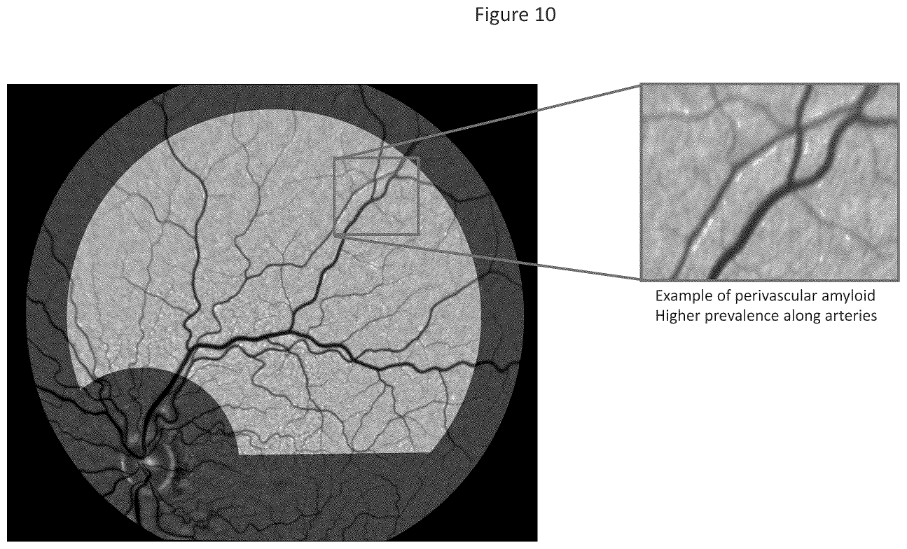

FIG. 11 demonstrates vessel tortuosity patterns characteristic of CAA+ subjects, with magnified inset highlighting retinal vessel tortuosity;

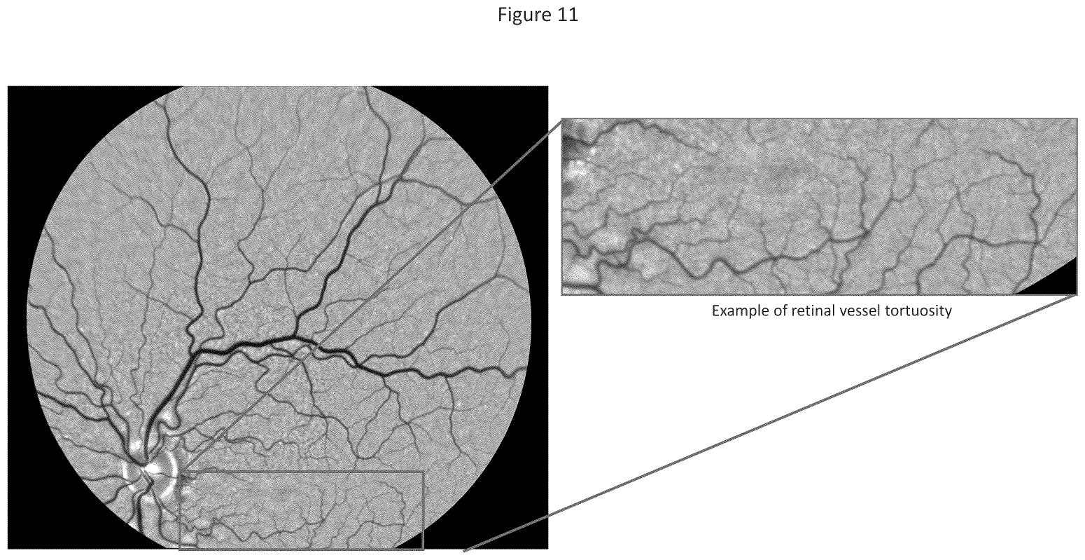

FIG. 12 shows both retinal hemorrhage and vessel tortuosity in a CAA+ subject, with magnified inset detailing these features;

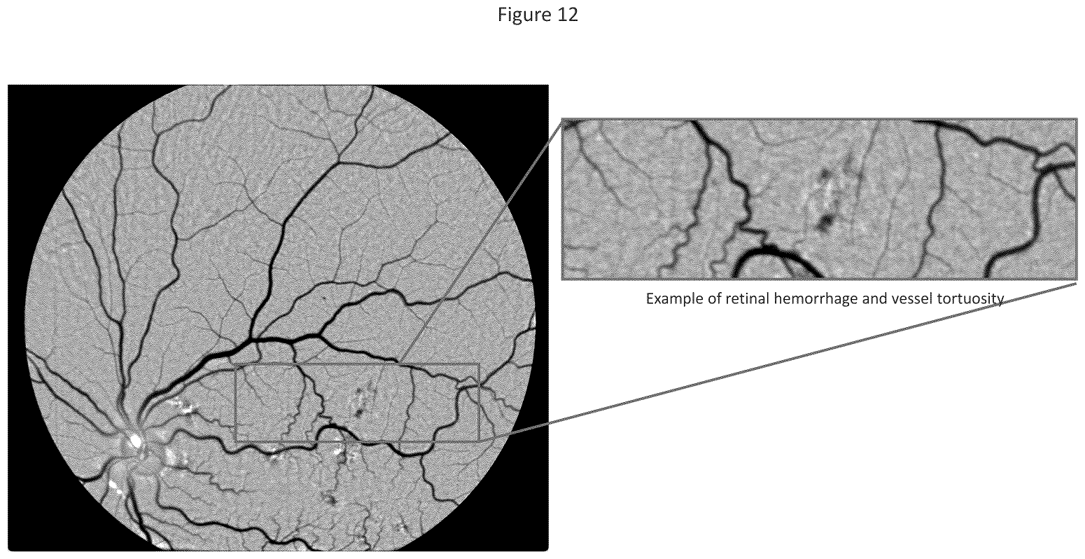

FIG. 13 presents a data table showing CAA prediction parameters, including features, descriptions, and area under curve (AUC) values for 29 subjects (5 CAA+ and 24 CAA−);

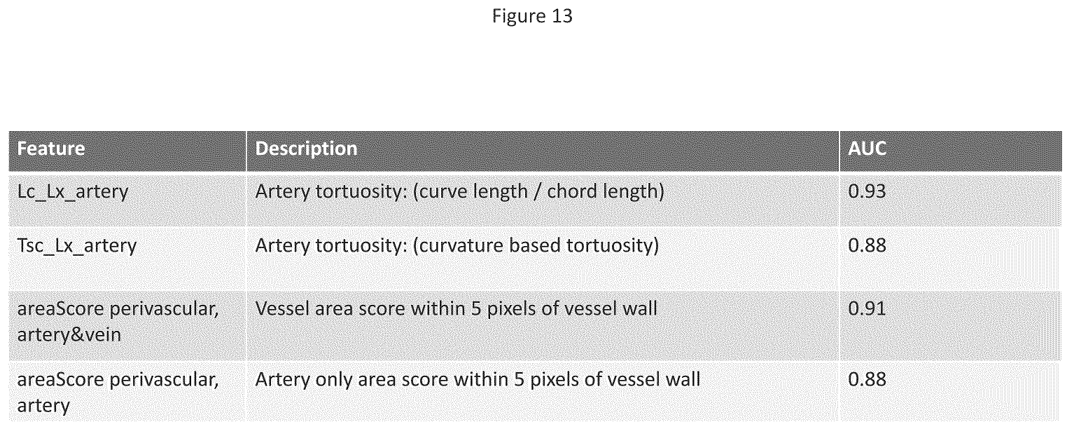

FIG. 14 illustrates the complete system for brain amyloid beta assessment using retinal imaging, incorporating high-resolution autofluorescent retinal camera and cloud-based software components.

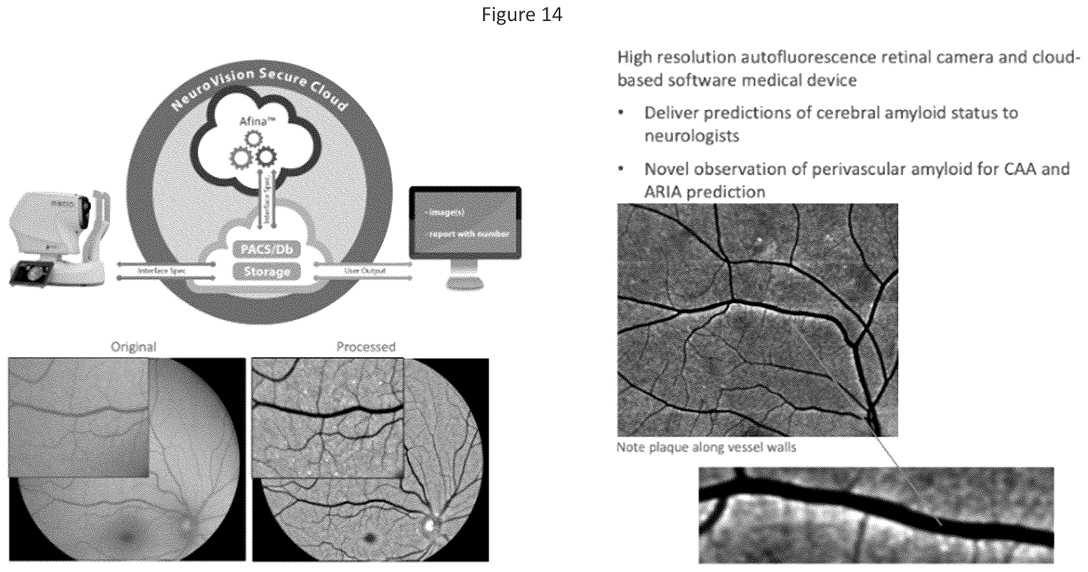

### DETAILED DESCRIPTION OF THE PREFERRED EMBODIMENT

The invention provides a method for detecting cerebral amyloid angiopathy (CAA) in a subject, comprising: capturing multiple autofluorescence images of the subject's retina; analyzing the images to detect and quantify perivascular amyloid accumulation along retinal blood vessels; and determining a likelihood of CAA based on the detected perivascular amyloid accumulation.

The method may further comprise creating a standardized region of interest using a registration function and detection of optic nerve head and fovea. The method may further comprise applying an image quality filter to eliminate low quality images. The method may further comprise detecting retinal hemorrhage and vessel tortuosity. The method may further comprise detecting retinal edema using optical coherence tomography (OCT).

The invention provides a method for detecting a cerebral amyloid angiopathy (CAA) condition of a subject, comprising: capturing multiple images of a retina of the subject; creating a standardized region of interest of the multiple images using a registration function, image quality assessment, and detection of optic nerve head (ONH) and fovea; applying an image filter and blink detector to eliminate images that are of low image quality; performing background correction on the images; applying a vessel detection algorithm on the images; applying a probability density function (PDF) fit of the retina and segmentation of retinal auto fluorescence; detecting any retinal hemorrhage and retinal vessel tortuosity; detecting any retinal edema by optical coherence tomography (OCT), and using individual elements and a combined data vector to predict the likelihood of CAA of the subject

The method may further include using the individual elements and combined data vector to predict the likelihood of Amyloid Related Imaging Abnormalities (ARIA-E and ARIA-H). The method may capture multiple images of a retina in blue auto fluorescence (AF), green AF, color, infrared (IR), and OCT The method may include in the step of applying an image filter and b link detector the step of eliminating images of low image quality due to cataracts and lid obstructions. The method may include steps being performed in a camera which captures multiple images. The images may be captured using an Optos wide field retinal imaging device. The method may include the step of quantifying amyloid along blood vessels. The method may include using a Center Vue Eidon or Heidelberg Spectralis device.

The invention provides a system for detecting cerebral amyloid angiopathy (CAA) in a subject, comprising: an image capture device for capturing multiple autofluorescence images of the subject's retina; a processor for analyzing the images to detect and quantify perivascular amyloid accumulation along retinal blood vessels, and determining a likelihood of CAA based on the detected perivascular amyloid accumulation.

The processor may create a standardized region of interest using a registration function and detection of optic nerve head and fovea. The system may further comprise an image quality filter to eliminate low quality images. The processor may detect retinal hemorrhage and vessel tortuosity. The processor may detect retinal edema using optical coherence tomography (OCT).

The invention provides a system for detecting a cerebral amyloid angiopathy (CAA) condition of a subject, comprising: an image capture device to capture multiple images of a retina of the subject; a processor for: creating a standardized region of interest of the multiple images using a registration function, image quality assessment, and detection of optic nerve head (ONH) and fovea; filtering the images and detecting blink to eliminate images that are of low image quality; performing background correction on the images; applying a vessel detection algorithm on the images; applying a probability density function (PDF) fit of the retina and segmentation of retinal auto fluorescence; and detecting any retinal hemorrhage and retinal vessel tortuosity; and an optical coherence tomography (OCT) device for detecting any retinal edema; and wherein the processor uses individual elements and a combined data vector to predict the likelihood of CAA of the subject.

The processor may use individual elements and combined data vector to predict the likelihood of Amyloid Related Imaging Abnormalities (ARIA-E and ARIA-H). The image capture device may capture multiple images of a retina in blue auto fluorescence (AF), green AF, color, infrared (IR), and OCT The processor may eliminate images of low image quality due to cataracts and lid obstructions. The processor may be part of a camera which captures multiple images. The image capture device may be an Optos wide field retinal imaging device. The processor may quantify amyloid along blood vessels. The image capture device may be a Center Vue Eidon or Heidelberg Spectralis device

A preferred embodiment will be described, but the invention is not limited to this embodiment.

In a preferred embodiment, multiple images of the retina are captured in blue AF, green AF, color, IR, and optical coherence tomography (OCT). The next step is a registration function, image quality assessment, automated detection of optic nerve head and fovea to create a standardized region of interest. Next, an image quality filter and blink detector is applied to eliminate frames that are of low image quality (cataracts), lid obstructions, etc. Next, a background correction is performed followed by automated vessel detection algorithm, followed by a probability density function (PDF) fit of the retina and segmentation of retinal autofluorescence. Next, segment autofluorescence that is adjacent to detected blood vessels is done. Then, detection of retinal hemorrhage and retinal vessel tortuosity is performed. Then, retinal edema is detected via OCT. Then, individual elements and a combined data vector are used to predict likelihood of CAA and also Amyloid Related Imaging Abnormalities (ARIA-E and ARIA-H).

Current retinal AF devices do not provide images and a signal that allow for measurement of these parameters, nor do they provide an image processing tool chain as described above to perform quantitative fluorescence in the region of interest, and/or along retinal blood vessels.

One novel aspect of the invention is the visualization and detection of perivascular amyloid via the capture of multiple frames and image processing tool chain as described above.

The invention can be used for the detection of perivascular amyloid, retinal vessel tortuosity, retinal hemorrhage, and retina edema for the quantification, prediction and disease management of CAA, ARIA, AD, and other neurological and cerebrovascular and retinal disorders. Furthermore, the invention can be used for quantification of AF/amyloid pre and post-treatment with monoclonal antibodies (and other treatments) regarding amyloid quantification and clearance as a response to therapy. The invention can also be used for prediction of cerebral amyloid status, disease state, cognitive status, vascular status, response to treatment, for (including but not limited to) CAA, ARIA, AD, and other neurological and vascular disorders.

The method and system of the invention may provide the following features:
1. Retinal edema and hemorrhage via OCT can be used as a predictor of CAA, ARIA, and AD either in clinical or home-based OCT devices.
2. The processing may be performed in a processor, which may be in the camera.
3. Can be used in the diagnosis and treatment of patients at risk for, or who have had stroke or hemorrhage.
4. Can be used to quantify the vascular contribution to neurological disorders in order to assist with diagnosis and response to therapy.
5. OCT parameters and measurements such as retinal nerve fiber layer thinning and other changes to the inner and outer retina, can predict (alone or in combination with other factors) CAA, ARIA, AD, and other neurological and vascular disorders including stroke and hemorrhage.
6. The images may be captured on Optos wide field imaging devices (including blue AF, green AF, OCT, color, and FA, and ICG modalities.)
7. The retro mode of a Nidek Mirante (and other related devices) may be used to quantitate amyloid along blood vessels via retro-illumination techniques.
8. The images may be acquired via the Center Vue (iCare) Eidon or Heidelberg Spectralis.

The registration function refers to a computational process that aligns multiple images of the same retinal area taken at different times or using different imaging modalities. This alignment ensures accurate comparison and analysis across images.

Image quality assessment involves automated evaluation of image characteristics including clarity, contrast, and signal-to-noise ratio to ensure reliable analysis.

The optic nerve head (ONH) and fovea detection utilizes specialized algorithms to automatically identify these key anatomical landmarks, which serve as reference points for standardized analysis.

The blink detector comprises software that identifies and flags images captured during eye blinks, which typically show partially or fully obscured views of the retina.

Background correction refers to computational processes that normalize image intensity and remove artifacts or noise from the background of retinal images.

The PDF fit involves statistical modeling of retinal autofluorescence patterns to identify areas of abnormal signal intensity.

Segmentation of retinal autofluorescence involves automated separation and classification of different retinal regions based on their fluorescence characteristics.

The data vector comprises a mathematical representation combining multiple measured parameters including vessel tortuosity, perivascular amyloid accumulation, hemorrhage presence, and edema measurements.

A preferred embodiment has been described, but variations will occur to those skilled in the art, and the invention is not limited to this embodiment. The scope of the invention is defined by the claims.

1. A method for detecting cerebral amyloid angiopathy (CAA) in a subject, comprising:
capturing multiple autofluorescence images of the subject's retina;
analyzing the images to detect and quantify perivascular amyloid accumulation along retinal blood vessels; and
determining a likelihood of CAA based on the detected perivascular amyloid accumulation.

2. The method of claim 1, further comprising:
creating a standardized region of interest using a registration function and detection of optic nerve head and fovea.

3. The method of claim 1, further comprising:
applying an image quality filter to eliminate low quality images.

4. The method of claim 1, further comprising:
detecting retinal hemorrhage and vessel tortuosity.

5. The method of claim 1, further comprising:
detecting retinal edema using optical coherence tomography (OCT).

6. A method for detecting a cerebral amyloid angiopathy (CAA) condition of a subject, comprising:
i. capturing multiple images of a retina of the subject;
ii. creating a standardized region of interest of the multiple images using a registration function, image quality assessment, and detection of optic nerve head (ONH) and fovea;
iii. applying an image filter and blink detector to eliminate images that are of low image quality;
iv. performing background correction on the images;
V. applying a vessel detection algorithm on the images;
vi. applying a probability density function (PDF) fit of the retina and segmentation of retinal auto fluorescence;
vii detecting any retinal hemorrhage and retinal vessel tortuosity;
viii. detecting any retinal edema by optical coherence tomography (OCT)
ix. using individual elements and a combined data vector to predict the likelihood of CAA of the subject.

7. The method of claim 6, further including using the individual elements and combined data vector to predict the likelihood of Amyloid Related Imaging Abnormalities (ARIA-E and ARIA-H).

8. The method of claim 6, wherein the capturing multiple images of a retina captures images in blue auto fluorescence (AF), green AF, color, infrared (IR), and OCT.

9. The method of claim 6, wherein the step of applying an image filter and blink detector eliminates images of low image quality due to cataracts and lid obstructions.

10. The method of claim 6, wherein the steps a-h are performed in a camera which captures multiple images.

11. The method of claim 6, wherein step a is performed using an Optos wide field retinal imaging device.

12. The method of claim 6, further including the step of quantifying amyloid along blood vessels.

13. The method of claim 6, wherein step a is performed using a Center Vue Eidon or Heidelberg Spectralis device.

14. A system for detecting cerebral amyloid angiopathy (CAA) in a subject, comprising:
An image capture device for capturing multiple autofluorescence images of the subject's retina;
A processor for analyzing the images to detect and quantify perivascular amyloid accumulation along retinal blood vessels, and determining a likelihood of CAA based on the detected perivascular amyloid accumulation.

15. The system of claim 14, wherein the processor:
creates a standardized region of interest using a registration function and detection of optic nerve head and fovea.

16. The system of claim 14, further comprising:
an image quality filter to eliminate low quality images.

17. The system of claim 14, wherein the processor:
detects retinal hemorrhage and vessel tortuosity.

18. The system of claim 14, wherein the processor:
detects retinal edema using optical coherence tomography (OCT).

19. A system for detecting a cerebral amyloid angiopathy (CAA) condition of a subject, comprising:
an image capture device to capture multiple images of a retina of the subject;
a processor for:
i. creating a standardized region of interest of the multiple images using a registration function, image quality assessment, and detection of optic nerve head (ONH) and fovea;
ii. filtering the images and detecting blink to eliminate images that are of low image quality;
iii. performing background correction on the images;
iv. applying a vessel detection algorithm on the images;
v. applying a probability density function (PDF) fit of the retina and segmentation of retinal auto fluorescence; and
vi. detecting any retinal hemorrhage and retinal vessel tortuosity; and
an optical coherence tomography (OCT) device for detecting any retinal edema; and
wherein the processor uses individual elements and a combined data vector to predict the likelihood of CAA of the subject.

20. The system of claim 19, wherein the processor uses individual elements and combined data vector to predict the likelihood of Amyloid Related Imaging Abnormalities (ARIA-E and ARIA-H).

21. The system of claim 19, wherein the image capture device captures multiple images of a retina captures images in blue auto fluorescence (AF), green AF, color, infrared (IR), and OCT.

22. The system of claim 19, wherein the processor eliminates images of low image quality due to cataracts and lid obstructions.

23. The system of claim 19, wherein the processor is part of a camera which captures multiple images.

24. The system of claim 19, wherein the image capture device is an Optos wide field retinal imaging device.

25. The system of claim 19, wherein the processor quantifies amyloid along blood vessels.

26. The system of claim 19, wherein the image capture device is a Center Vue Eidon or Heidelberg Spectralis device.
